(19) United States
(12) Patent Application Publication
(10) Pub. No.: US 2023/0310245 A1
(43) Pub. Date: Oct. 5, 2023

(54) PROTECTION DEVICE FOR PROTECTING AGAINST PATHOGENS, AND METHOD

(71) Applicant: SPHAIRA MEDICAL GMBH, Berlin (DE)

(72) Inventors: Janis Muench, Berlin (DE); Moritz Eichhorn, Berlin (DE)

(21) Appl. No.: 18/014,935

(22) PCT Filed: Jul. 8, 2021
(86) PCT No.: PCT/EP2021/069020

§ 371 Date: Jan. 6, 2023

(30) Foreign Application Priority Data
Jul. 8, 2020 (DE) 10 2020 118 071.3

(51) Int. Cl. A61G 10/00; A61G 10/02

(52) U.S. Cl. CPC A61G 10/005; A61G 10/023

(57) ABSTRACT

A protective device to protect against pathogens comprises a protective housing that defines an interior space defined, a closable access opening to enter the interior space, a seating element that allows a passenger to sit in the interior space, and a chassis to move the protective device, wherein the protective device, in a closed state, seals the interior space in an airtight manner against an environment. A method to protect against pathogens comprises providing a protective device to a passenger, closing the protective device in an airtight manner, and moving the protective device. The protective device and/or the method may be used to protect a patient.

The present disclosure relates to a protective device to protect against pathogens.

Known protective devices, for example chemical protection suits or other personal protective wear, are cumbersome and time-consuming to use. In an example, infants, seniors, sick and disabled person may thus not be able to use protective devices at all.

It is thus an object of the present disclosure to provide a protective device that reduces or removes the previously mentioned disadvantages. In particular, it is an object of the present disclosure to provide a protective device, which is simple and timesaving to use.

This problem is solved by a protective device to protect against pathogens, comprising a protective housing that defines an interior space, a closable access opening to enter the interior space, a seating element that allows a passenger to sit in the interior space in a closed state of the access opening, and a chassis for moving the protective device, wherein the protective device seals the interior space in an airtight manner against an environment in a closed state of the access opening.

The protective device forms a protective interior space, in which a passenger may be. The protective device in particular protects against pathogens, i.e., for example bacteria, parasites, viruses, prions, toxins, fungi and toxic substances. In particular, the protective device protects against substances that may harm the health of a passenger, such as, for example, dust, in particular particulate matter, vapors, aerosols and the like.

For example, the protective device may protect the passenger against pathogens and/or substances from the environment of the protective device. If, for example, the passenger is a healthy visitor in a hospital, the passenger may be protected against pathogens that an infectious patient spreads in an environment of the protective device.

In another example, the protective device may protect the passenger against an environment that is adverse and/or hazardous to the health for the passenger, for example in an environment with a high air pollution, with a high dust and/or radiation contamination and/or with chemicals being present in the environmental air.

In another example, the protective device can protect persons that stay in the environment of protective device against pathogens and/or substances that are emitted by the passenger. If, for example, the passenger suffers from an infectious disease and/or the passenger is contaminated with substance that is hazardous to health, persons that visit may be protected against an infection during contact with the passenger. In an example where the passenger visits a patient with a weak immune system, the patient may be protected against an infection during a contact with the passenger.

The interior space of the protective device is defined by a protective housing that essentially, in particular fully, surrounds the interior space. The protective housing is sized in such a way to fully accommodate a seating passenger therein. The protective housing is furthermore small enough to allow a communication of the passenger with the environment of the protective device.

The protective housing completely closes the interior space. In an example, the protective housing may be configured in form of a capsule that fully encloses the interior space. The capsule may, in this case, form the cabin for the passenger at the same time.

In another example, the protective housing may be configured in the form of a shell, which is fully flush with a bottom element. In a further example, the protective housing may be configured in a two-part form, for example in the form of a frame and a lid that are flush with each other.

Due to the fact that the protective housing completely closes the interior space, the interior space may particularly protect in a versatile manner. In an example, the interior space may thereby protect against certain radiation. In a further example the interior space may thereby protect against aerogene pathogens, i.e. pathogens that are transmitted through the air. Thereby, an infectious patient may visit a weakened patient in a hospital.

Due to the fact that the protective housing defines the interior space, the protective device may have a particularly even surface with very little gaps. Thereby, the protective device may be cleaned, disinfected, decontaminated or sterilized easily and effectively.

The protective device comprises an openable and closable access opening to enter the interior space. The access opening can, for example, be configured as a door in the protective housing. In a further example, the access opening may be configured as a lid that forms, in a closed state, the protective housing together with a frame.

Due to the fact that the protective housing comprises an openable and closable access opening to enter the interior space, a passenger may enter the interior space easily and comfortably. In a closed state the access opening closes the protective housing in such a way that the protective device protects against pathogens.

The protective device further comprises at least one seating element, in particular exactly one seating element that allows a passenger, in particular exactly one passenger, to sit in the interior space. Due to the fact that exactly one passenger is allowed to sit in the interior space, the protective device may be configured particularly compact, such that it may be moved in narrow rooms.

Alternatively, the protective device comprises at least two seating elements, in particular exactly two seating elements. The two seating elements may be arranged after one another or next to each other, for example.

Due to the fact that exactly two seating elements are provided, exactly two persons may be allowed to sit in the interior space. These may, for example, be a passenger, in particular a passenger that is protected against pathogens, and an accompanying person, such as, for example, medical staff. Alternatively, the two seating elements may be taken by two passengers that visit a patient in the protective device, wherein the two passengers are protected against pathogens of the patient and/or wherein the patient is protected against pathogens of the visitors.

In particular, the exactly two seating elements may be configured as a smaller seating element and a larger seating element. Thereby, it is achieved that a child and an adult person may be passengers of the protective device, for example two relatives or a child and an accompanying person. Thereby, the protective device may be configured particularly compact.

If the two seating elements are provided after one another, it is achieved that the protective device may be configured particularly narrow and thereby may be moved through narrow passages.

If the two seating elements are provided next to each other, it is achieved that the passenger may be cared for particularly well as a direct access to the passenger may be realized through the medical staff. In addition, through such an arrangement, a particularly agile protective device is achieved.

Alternatively, the protective device comprises at least four seating elements, in particular exactly four seating elements. The four seating elements may be arranged as two seating elements next to each other in two rows after one another.

Due to the fact that exactly four seating elements are provided, a group of passengers may be allowed to use the protective device while the protective device is limited to compact outer dimensions. Therein the group of passengers may be passengers against which the environment is protected against pathogens, with which the passengers are infected. In particular, it is thereby possible that multiple passengers that are infected with the same pathogen can be transported. Alternatively, or additionally, one, two or the accompanying person may accompany the passenger or passengers.

Alternatively, the group of passengers may be visitors of a patient that are protected from pathogens of the patient and/or the patient is protected against pathogens of the visitors.

In a case where the protective device has exactly one seating element, the protective housing is, for example, more than 60 cm long, 50 cm wide and 80 cm high. In another example, the protective housing is less than 180 cm long, 150 cm wide and 200 cm high. In an example, the protective housing is between 100 cm and 140 cm long, between 80 cm and 120 cm wide and between 140 cm and 180 cm high.

In a case where the protective device has exactly two seating elements after one another, the protective housing is for example between 160 cm and 210 cm long, between 80 cm and 120 cm wide and between 140 cm and 180 cm high.

In a case where the protective device has exactly two seating elements next to each other, the protective housing is for example between 80 cm and 120 cm long, 160 cm and 210 cm wide and between 140 cm and 180 cm high.

In a case where the protective device has exactly four seating elements, two next to each other and behind one another, the protective housing is for example between 160 cm and 210 cm long, between 100 cm and 180 cm wide and between 140 cm and 180 cm high.

In one example the seating element or the seating elements are each configured as a seating shell. The seating shell can be flush with the protective housing or be integral with the protective housing.

Due to the fact that seating element is a seating shell, the protective device may be comfortably used and easily cleaned. Furthermore, the protective device may be configured in a compact manner when the seating element is configured as a seating shell.

In other examples the seating element may be configured as a stool, a chair, a bench or an armchair.

The seating element is configured to allow for a comfortable seating of the passenger. The seating element may be configured in a way that the seating surface of the seating element comprises a height between 45 cm and 95 cm to the bottom of the environment of the protective device.

Preferably the seating element comprises a raised seating surface, such that the passenger is positioned on eye level with a patient that is positioned at least partly upright in a hospital bed. In an example, the seating element thereby comprises a seating surface at a height between 60 cm and 80 cm from the bottom of the environment of the protective device.

Due to the fact that the seating element is configured in a way that the seating surface comprises a height between 60 cm and 80 cm to the bottom of the environment of the protective device, the head of the passenger is on eye level with a patient that is positioned in a hospital bed and the hands of the passenger are positioned on the same level as a patient lying in a hospital bed.

The seating element is preferably configured for an upright seating. In an example, an angle between the seating surface of the seating element and a back rest of the seating element or a wall of the protective housing may be between 80° and 110°, preferably between 90° and 100°.

Due to the fact that the seating element is configured for an upright seating, the face and/or the limbs of the passenger may reach particularly close to the protective housing. Thereby, the passenger can particularly easily and directly communicate and/or interact with one or multiple persons in the environment of the protective device.

The protective device comprises a chassis. The chassis comprises one or more wheels, for example solid rubber wheels, wheels with a combination of a tire and a rim, or plastic rollers. Furthermore, the chassis may comprise one or more wheel mountings, wheel bearings, brakes, suspensions and steering elements. One or more of the wheels may be mounted freely rotatable, for example by using a ball bearing. In an example, the chassis may comprise one or two freely rotatable mounted wheels and one or two steerable wheels. In another example the vehicle may comprise two steerable wheels and two fixedly arranged wheels.

Therein, the capsule of the protective device may be in particular fully integral with the chassis. Thereby, particularly compact outer dimensions are achieved. In particular, the whole protective device or all relevant components thereof may be integral. Thereby, a particularly compact design is achieved.

The protective device seals the interior space in a closed state of the access opening against an environment in an airtight manner. That means that essentially no uncontrolled exchange of air between the interior space of the protective device and the environment of the protective device happens or is possible.

The air tightness of the protective device is in particular based on a closing of the access opening in an airtight manner. The access opening comprises a hatch, which may also be identified as a door or a flap, and a receiving portion. In a closed state the hatch engages with the receiving portion. The hatch and/or the receiver comprise a seal, for example a rubber lip. In a closed state the hatch is pressed against the receiving portion, such that the seal does not allow for an exchange of air.

For example, the rubber lip may be attached in a frame. In an example, the rubber lip is made from ethylene propylene diene monomer rubber (EPDM). In another example the rubber lip is made from silicone. In an example, the rubber lip is made from rubber. Alternatively, the rubber lip is configured as an activatable, for example inflatable, profile.

In particular, the access opening allows for a direct access to the interior space, whereby the usage is easy and a particularly simple protection against pathogens is achieved.

The contact pressure force between the hatch and the receiving portion may be maintained by a lever lock that locks the access opening. Alternatively, or additionally, the contact pressure force between the hatch and the receiving portion may be pretensioned and afterwards maintained by a tension lock that locks the access opening when closing the access opening.

Due to the fact that the access opening is locked with a lock, in particular with a lever lock and/or a tension lock, the access opening may be easily locked in an airtight manner. Thereby, the protective device may be particularly effectively protect against pathogens.

Alternatively, or additionally, the contact pressure force between the hatch and the receiving portion may be maintained by a screw lock that locks the access opening.

Due to the fact that the access opening is locked with a screw lock, the contact pressure force may be particularly well regulated. Thereby, the protective device may be particularly secure.

Alternatively, the access opening may be locked by driving an electric motor that ensures a correct contact pressure force. Thereby, a particularly easy, secure, and repeatable locking is possible.

To ensure the air tightness and to check the protective function during operation, an air measurement sensor for checking the air may be provided in the inside of the protective capsule. The air measurement sensor may, for example, measure the pressure on the interior and exterior and, in response thereto control the electric motor that provides the contact pressure force.

Alternatively, the air measurement sensor may measure the pressure on the interior and exterior and control power of ventilation and deaeriation accordingly and/or create an alarm signal. In an example, the air measurement sensor may also measure the composition of the air on the interior of the capsule.

Furthermore, further sensors may be provided that are configured to measure various vital data, such as, for example, weight, heart rate, blood pressure, blood sugar, or oxygen saturation of the passenger.

Due to the fact that the protective device seals the interior space in a closed state of the access opening in an airtight manner against an environment, an exchange between the interior space of the protective device and the environment of the protective device may be controlled. In an example, the exchange may be temporarily suspended. In a further example only air from a secure part of the environment may be exchanged with the interior space, for example through a hose. Thereby, the protective device may particularly easily protect against pathogens.

According to an embodiment of the protective device, the protective housing consists at least partly of a thermoplastic material. Thermoplastic materials may consist of semi-crystalline polymers and, in this case, may be translucent (i.e. cloudy) or opaque (i.e. untransparent). Thermoplastic materials may consist of amorphous polymers and be, in this case, transparent (i.e. translucent).

Due to the fact that the protective housing at least partly consists of a thermoplastic material, the protective housing may be cheaply made and easily processed. Thereby, the protective device may be manufactured particularly economically.

If the protective housing at least partly consists of a thermoplastic material, the protective housing may be exchanged advantageously. Thereby, the protective device may be particularly hygienic.

The thermoplastic material may be, partly or fully, modified, e.g. tinted, coated or surface-treated. In an example, the thermoplastic material may be configured to absorb and/or reflect light of certain wavelengths. In a further example, the thermoplastic material may be, partly or fully, darkened, such that only a fraction of the light falling on the thermoplastic material reaches the interior space of the protective device. Therein, the thermoplastic material may be phototropic, i.e. the thermoplastic material may be adapted darken by itself at high brightness and become more transparent again when brightness decreases. In a further example, the thermoplastic material may be configured to absorb and/or reflect radiation, for example UV radiation, heat radiation, or radio radiation.

If the thermoplastic material is modified, the protective housing may absorb and/or reflect radiation, for example light, UV radiation, heat radiation, or radio radiation. Thereby, the passenger may be protected from further hazard sources, for example if the passenger is particularly sensitive or if the environment is particularly hazardous.

Due to the fact that the protective housing at least partly consists of a thermoplastic material, the protective housing may be particularly cheap, break-proof and lightweight. Thereby, the protective device may be particularly cheap and robust in acquisition and maintenance.

Further, by providing the protective housing at least partly from a thermoplastic material, the protective device may be at least partly transparent. Thereby, the protective device may allow for a particularly personal communication, for example through facial expressions or gestures, between the passenger and a person in the environment of the protective device. Furthermore, the passenger may particularly easy and fully perceive the environment of the protective device, for example obstacles. Thereby, the protective device may be particularly secure.

The protective device may be improved in that the protective housing consists at least partly from acrylic glass. Acrylic glass is a transparent thermoplastic material. The protective housing may, for example, completely consist of acrylic glass, consist of acrylic glass except individual elements, or have windows from acrylic glass.

Due to the fact that the protective housing consists at least partly from acrylic glass, the protective housing may be particularly transparent. Thereby, the protective housing may particularly enhance a communication between the passenger and other persons.

Furthermore, a transparent embodiment allows for a better interpersonal contact between the passenger and persons in the environment, whereby the perception of an isolation situation is further reduced.

Acrylic glass is particularly resistant against many chemical agents. For example, acrylic glass is particularly resistant against acids, lyes of medium concentration, gasoline and oil. Furthermore, acrylic glass is very sturdy against mechanical impact. Thereby, the protective housing may protect the interior space particularly versatile. Thereby, the protective device may be particularly secure.

Furthermore, because the protective housing consists at least partly of acrylic glass, the protective housing is particularly smooth. Thereby, the protective housing may be particularly effectively cleaned, disinfected, and sterilized. Thereby, the protective device may be particularly hygienic and economically provide a particularly effective protection.

The acrylic glass may have a scratch-resistant coating. Thereby, the optical properties of the acrylic glass may be maintained longer. Thereby, the can the value and durability of the protective device may be improved.

Alternatively, the protective housing can consist partially or fully from glass, in particular silicate glass. Glass absorbs UV light that may be potentially harmful for the passenger. Furthermore, glass is particularly smooth and thereby particularly hygienic, as germs may not easily attach thereto and as it may be easily cleaned and disinfected.

Due to the fact that the protective housing is made of glass, the protective housing may have very good optical properties. Thereby, the communication of the passengers with its environment may be improved.

According to an embodiment of the protective device, the chassis comprises an electrical drive and the protective device comprises an electrical storage element that is configured to provide energy to the electrical drive.

In one example, the electrical drive comprises an electric motor that is connected to an axle. In a further example, the electrical drive comprises at least one electric motor that drives a wheel. The electrical drive can further comprise an electrical steering.

The electrical drive may be configured such that the protective device is adapted to accelerate to a speed of up to 6 km/h. In this case, the protective device may be particularly easy and safe to operate. Alternatively, the electrical drive may be configured such that the protective device is adapted to accelerate to a speed of up to 15 km/h. In this case, the protective device may cover longer distances quickly and achieve a high utilization.

Alternatively, the electrical drive may for example be configured such that the protective device is adapted to accelerate to a speed of up to 25 km/h. In this case the protective device may allow for coverage of far distances outside of buildings.

Due to the fact that the chassis comprises an electric drive, the protective device may be moved particularly cheap, autonomous, quiet, emission-free and secure. Thereby, the protective device may be operated particularly cheap and versatile.

The electrical storage element is configured to provide electrical energy. The electrical storage element may, for example, comprise a battery, preferably a secondary battery, that may also be identified as an accumulator. The electrical storage element may, in another example, comprise a fuel cell system. The fuel cell system can comprise, for example, a hydrogen tank and a hydrogen-oxygen fuel cell.

Due to the fact that the protective device comprises an electrical storage element that is configured to supply the electric drive with energy, the protective device may be moved particularly autonomous and versatile, for example across far distances and/or outside of buildings.

Thereby, the protective device may, for example, be employed on a hospital campus that encompasses multiple hospital buildings. In an example, the protective device may in this case store energy, for example load an accumulator, in a first hospital building and, using the stored energy, move to a second hospital building, move inside there and move back to the first hospital building.

In an embodiment, the protective device is configured hygienic.

Hygienic configuration identifies a design that counteracts a contamination, i.e. that reduces the probability of a contamination, and eases cleaning, disinfection or sterilization.

The protective device therefor may consist at least partly of hygienic materials. In an example, the materials may predominantly comprise inert substances. Inert substances do not react or react only under certain conditions with potential reactants. In a further example, the materials may exclusively be non-toxic. In a further example, the materials may exclusively comprise materials are stable across a certain temperature spectrum. As an example material, stainless steel basically fulfills such requirements.

The protective device may have a hygienic setup. In an example, the areas employed in the protective device may be configure substantially smooth and without surface roughness or cavities. The areas and connections may be arranged such that the protective device does not comprise protruding parts, bars or hidden corners. Thereby, organic substances may not attach to the areas and connections.

Further, the protective device may be constructed, for example, without dead spaces, i.e. spaces that are difficult to clean. In an example, potential dead spaces may be avoided in that one or more elements of the protective device are configured to be easily dismantled for cleaning.

The construction may be configured such that possible service openings, for example, to access one or more electric motors or the electrical storage element from a bottom side of the protective device, are closed against the environment in an airtight manner. Thereby, cleaning after usage is eased.

In another example, the protective device may comprise drains and beads, such that an unhindered flow of disinfectants in the whole of the protective device is ensured.

Due to the fact that the protective device is designed in a hygienic manner, the protective device may be made operatable particularly quickly and may be operated particularly securely.

According to an embodiment of the protective device, the protective housing comprises an anti-bacterial and/or an anti-viral coating. In an example, the protective device may have an anti-bacterial and/or an anti-viral coating on an interior side of the protective housing and/or on an exterior side of the protective housing. Alternatively, or additionally, the seating element and/or further elements of protective device may also comprise an anti-bacterial and/or an anti-viral coating.

Because the protective housing comprises an anti-bacterial and/or an anti-viral coating, germs may be neutralized before they spread further or multiply. Thereby, the protective device may be operated particularly secure.

In an embodiment, the protective device comprises a ventilation element that is configured to provide an air stream to the interior space.

In an example, the ventilation element may comprise an air reservoir, for example a compressed air reservoir or an oxygen reservoir, that allows for an air supply to the interior space that is independent from the environment. This air reservoir consists, for example, of cleaned and checked air, which is free from pathogens.

In an example, the ventilation element may be attachable to an air duct, for example to an air duct of a building, for example an air duct of a hospital. The ventilation element may receive clean air from the air duct that is independent from the air in the environment of the protective device and provide it to the interior space. Alternatively, or additionally, the ventilation element may be configured to discharge, via the air duct, potentially contaminated air from the interior space of the protective device, without risking contaminating the environmental air.

In an example, the ventilation element may be configured, to receive environmental air, to process it and to provide it to the interior space. Alternatively, or additionally, the ventilation element may be configured to receive potentially contaminated air from the interior space of the protective device, to process it, and to release to release it to the environment. Processing may comprise filtering, e.g. through a HEPA filter. Processing may, additionally, or alternatively, comprise degerminate using UV-C radiation. Processing may, additionally, or alternatively, comprise disinfecting using ozone gas in connection with UV-C radiation. Processing may, additionally, or alternatively, further comprise steps for cleaning, disinfecting, sterilizing or degerminating the air.

Due to the fact that the protective device comprises a ventilation element that is configured for providing an air stream to the interior space, the interior space may be supplied with air without requiring a direct air exchange with the environmental air. Thereby, a passenger may be in the interior space for an extended period of time, without being exposed to a contamination by the environment and without exposing the environment to a contamination. Thereby, the protective device may be particularly secure.

According to an embodiment of the protective device the ventilation element is configured to create an overpressure and/or a negative pressure in the interior space.

In an example, the ventilation element may supply more air to the interior space than it discharges air from the interior space, to create an overpressure in the interior space. An overpressure in the interior space may additionally prevent an uncontrolled entering of potentially contaminated air from the environment into the interior space. Thereby, a passenger may be particularly well protected.

In an example, the ventilation element may supply to the interior space less air than it discharges from the interior space, to create a negative pressure in the interior space. A negative pressure in the interior space may additionally prevent an uncontrolled exiting of potentially contaminated air from the interior space to the environment. Thereby, the environment may be particularly well protected.

According to an embodiment of the protective device the ventilation element is configured to supply an air-conditioned air stream to the interior space.

For example, the air-conditioned air stream may be tempered, i.e. cooled or heated. In a further example, the can air-conditioned air stream may be conditioned, i.e. dried or humidified.

Due to the fact that the ventilation element is configured to provide an air-conditioned air stream to the interior space, it is possible to ensure a particularly passenger-friendly atmosphere in the interior space. Thereby, for example, a sensitive passenger may stay in the protective device for an extended time period, without suffering discomfort.

In an embodiment, the protective device is configured to be remote-controlled. The protective device comprises a control element that is configured to remote-control the protective device.

The control element can comprise one or more control devices. The control element can be fully or partially configured as software.

In an example, the chassis may, for example an electrical drive, may be remote-controlled. In an example, the chassis may be remote-controlled by a person that is directly in the line of sight of the protective device, for example by a nurse. In a further example, the chassis may be remote-controlled by a person that has vision of a driveway of the protective device by use of a camera, for example by a service employee in a control room distanced from the protective device, such as, for example, a call center. In a further example, the protective device may be remote-controlled by a computer.

Due to the fact that the protective device may be remote-controlled, the protective device may be moved without the requirement of a person accompanying the protective device, and, at the same time, without assigning responsibility for the movement to a passenger. Thereby, the protective device may be used more versatile and securely.

In a further example, the ventilation element may be remote controlled. In an example, the ventilation element may be switched on, switched off or adjusted. Thereby, it is possible, to adjust the supply of a particularly cleaned air stream when entering a secure area, and/or to adjust a more simple air stream when leaving the secure area.

In a further example, a disinfection element, as it will be described in the following, may also be remote controlled.

According to an embodiment, the protective device may comprise a disinfection element that is configured to disinfect the protective device.

The disinfection element is configured to disinfect a part of the protective device or the whole protective device.

The disinfection element can therefor comprise one or more jets that are connected with a disinfection reservoir. The disinfection element can provide a disinfectant from the disinfection reservoir to the jets, whereby the disinfectant is sprayed. The disinfection element preferably applies the disinfectant to the interior space. Additionally, or alternatively, the disinfection element may be configured to apply disinfectant to the exterior of the protective device, in particular to the exterior of the protective housing. Additionally, or alternatively, the disinfection element may be configured, to apply disinfectant in an area of the access opening.

In an example, the disinfection element may be configured to apply disinfectant manually, for example by using a pump device and a flexibly movable jet.

In a further example, the disinfection element may be configured to apply disinfectant mechanically, for example in a predefined angle and/or by a mechanical rotation of the jets. The disinfectant thereby may be sprayed continuously while the jets are turned, pivoted or moved. Thereby, a quick disinfection of a large area of the protective device may be achieved. In an example, the disinfectant may be sprayed periodically, i.e. in a pumping sequence. Thereby, an economical application of the disinfectant is achieved. Application of a disinfectant may be triggered remote-controlled or at the touch of a button.

In a further example the disinfection element may be configured to apply disinfectant automatically. Application of disinfectant may be triggered based on a predefined schedule, upon opening of the access opening and/or on a determined, estimated or calculated degree of contamination.

Due to the fact that the die protective device comprises a disinfection element that is configured to provide a disinfectant, the protective device may be maintained permanently in a secure and hygienic state. Thereby, the protective device is more secure and cheaper to operate.

In an embodiment, the protective device is configured for autonomous driving.

For example, the protective device may autonomously head for a preselected room in a hospital. Therein, the protective device may avoid collisions, evade obstacles, and reach the destination autonomously. Autonomous driving may, for example, be executed by a driving computer, that is a part of the protective device. Alternatively, or additionally, autonomous driving may be executed by a network service that is provided to the protective device via a network, for example via a radio network.

Due to the fact that the protective device is configured for autonomous driving, the protective device may allow for mobility of the passenger without having to rely on further persons. Thereby, the protective device may be operated cheaper and more versatile.

In an embodiment, the protective device further comprises a control element. The control element can for example comprise a touch display or a joystick. The control element may, for example, be configured to receive a selection of a driving destination and to provide it to a driving computer. The control element can further be configured to provide control of the ventilation element, the disinfection element or opening and/or closing of the access opening.

The control element can for example be arranged in the interior space of the protective device arranged, for example at the only seating element, such that a passenger of the protective device may control it himself. Thereby, a passenger may autonomously control the functions of the protective device, without having to rely on a further person. Thereby, the protective device may be operated cheaper and more versatile.

Alternatively, or additionally, the control element may be arranged at one of exactly two or exactly four seating elements, for example, in particular exclusively, at the front or rear oft wo seating elements being arranged behind one another. Thereby, an accompanying person may control the protective device, in particular when the passenger requires aid.

Alternatively, or additionally, the control element may be arranged at the outer side of the protective device, such that an external person may control the protective device from the outside.

According to an embodiment of the protective device, the chassis is configured to be off-road capable. In an example, the chassis may have wide tires with all-terrain profile. Furthermore, the chassis may have a high ground clearance, for example a ground clearance of more than 10 cm, for example more than 20 cm, in particular about at least 40 cm.

Due to the fact that the chassis is capable for off-road, the protective device may be used particularly versatile, for example also in disaster zones and in ad-hoc hospitals. Thereby, the protective may be used more versatile.

According to an embodiment of the protective device, the protective housing comprises flexible pass-through elements. The pass-through elements can for example be configured as invertible gloves. Invertible gloves may be easily inverted, such that they can be worn from a side of the passenger as well as from a side of the environment.

In an example, the pass-through elements may comprise single use gloves. The single use gloves may be configured to be directly attached to the protective housing, wherein the protective device remains airtight when the single use gloves are attached. The single use gloves may alternatively configured to be attached to the protective housing by using glove coupling elements, wherein the protective device is airtight, when the single use gloves are attached. The single use gloves can alternatively be configured to be inserted into gloves fixedly attached to the protective housing, wherein the protective device remains airtight, even without the single use gloves.

Due to the fact that the pass-through elements comprise single use gloves, hygiene may be particularly easily ensured. Thereby, the protective device may be particularly safe.

Due to the fact that the protective housing comprises flexible pass-through elements, the passenger and a person being in the environment of the protective device may easily exchange touches and gestures, without the risk of a contamination. Thereby, the protective device may allow for and improve interpersonal communication.

The material of the gloves can be configured from a particularly skin-friendly and haptic material, which is at the same time airtight. The material of the gloves may, for example, be made from a TPU-coated nylon fabric. In a further example, the material may be made from polyethylene fibers (HDPE, high-density polyethylene).

For example, the material may consist of multiple layers, wherein the outer layers convey a haptically pleasant feeling and the inner layers are airtight or sealed against the exchange of pathogens.

For example, the inner layer may be formed from latex rubber or plastics and the outer layers may be made from silk or other textile fibers. In an embodiment the inner and outer layer may be embodied as a flocked material.

Thereby, the haptic and human touch and perception is improved and the isolation situation is improved.

In an embodiment, the protective device further comprises an emergency element.

In an example, is the emergency element is configured as an emergency handle. The emergency handle may be configured to open the access opening of the protective device. The emergency handle may alternatively be configured to create an air supply between the interior space and the environment, for example a to open a roof hatch of the protective device. The emergency handle may alternatively be configured to cause an emergency stop of the chassis, e.g. by blocking one or more of the wheels.

In a further example the emergency element is configured as an emergency switch. The emergency switch may, for example, switch off an electric drive of the chassis, switch off processing of air by the ventilation element, release a lock of the access opening, initiate a communication, or trigger an alarm. The alarm may be triggered in a system, for example via mobile communication or a radio network in a hospital alarm system. The alarm may alternatively be triggered directly at the protective device, for example via acoustic signals, such as a siren, and/or optical signals, such as a warning light.

Due to the fact that the protective device comprises an emergency element, the passenger may react to critical or dangerous situations. Thereby, the protective device may be configured more secure.

According to an embodiment, the protective device comprises medical equipment.

The protective device may, for example, comprise a ventilator device, to support breathing of the passenger or to ventilate him. The protective device may comprise measuring devices, for example heart rate measuring devices, blood pressure measuring devices, or breath measuring devices. The protective device may be equipped with further medical equipment.

Furthermore, the protective device may comprise fastening devices in the form of hooks and loops, to which medical devices, such as infusions or patient measuring devices, may be attached.

As an example, fastening devices may be made from plastic. In another example, the fasteners may be made from metal. In an example, the fastening devices may be arranged in the interior of the protection space, in particular in an upper part, in a head area of the passenger or above, and/or in a lower part, in a foot area of the passenger or below.

Due to the fact that the protective device comprises medical equipment, a stay of a passenger in the protective device for an extended period of time is allowed. Thereby, the protective device may be used more versatile.

In an embodiment, the protective device further comprises a intercom system. The intercom system may comprise speakers and/or microphones on the exterior of the protective device and/or in the interior space of the protective device. The intercom system may alternatively be configured in the form of an acoustical bridge that does not require electrical energy.

Due to the fact that the protective device comprises an intercom system, the passenger may communicate better and easier with one or more persons in the environment of the protective device, for example with a visited patient, or—in case one of the participants is hard of hearing—may communicate at all.

In an embodiment of the protective device, the seating element has a seating surface that is adjustable in height. The seating surface may be electrically adjustable in height, for example via a linear drive. The seating surface may also be mechanically adjustable in height, for example hydraulically.

Due to the fact that the seating element has a seating surface that is adjustable in height, the passenger may communicate particularly well and comfortably with a person in the environment of the protective device, for example with a patient that is restricted in the freedom of movement. Thereby, the protective device may be used more comfortable and versatile.

According to a further aspect of the present disclosure, the initially mentioned problem is solved by a method, comprising providing a protective device, in particular a protective device according to one of the previously described examples, to a passenger, closing, in an airtight manner, of the protective device, and moving the protective device. The protective device may be in particular a protective device according one of the embodiments as described above.

Providing of the protective device in particular takes place in a secure environment. If, for example, the protective device is supposed to protect a patient that is visited by a passenger of the protective device, providing the protective device takes place in an area out of reach of the patient or in a secure distance outside of the infection radius of the patient. If, for example, the protective device is supposed to protect the passenger against pathogens, providing the protective device takes place in a sterile area, for example in a cleanroom.

Due to the fact that providing takes place in a secure environment, the method may reliably protect against pathogens.

Closing the protective device in an airtight manner comprises closing the access opening. Closing the protective device in an airtight manner may comprise pressing a hatch of the access opening against a seal, for example a rubber lip, and locking the hatch.

Thereby, the access opening may be locked particularly in an airtight manner.

Locking may take place from the exterior of the protective device. In this case, the protective device may be configured to be unlocked an opened from the interior of the protective device in case of an emergency.

Thereby, trained staff, for example hospital staff, may conduct the locking. Thereby, the protective device may be closed particularly reliable in an airtight manner.

Moving the protective device may take place by pushing or pulling the protective device, for example by hospital staff. In this case, the protective device does not comprise an own motoric drive, in particular no electric drive. Alternatively, or additionally, moving the protective device may happen via an electric drive. The electrical drive may for example facilitate pushing or pulling in form of an auxiliary drive. In particular, the electrical drive may in this case support pushing or pulling, i.e. the protective device is only driven and in thus far as the protective device is pushed or pulled. Alternatively, or additionally, the electrical drive may allow for an autonomous movement, for example a remote-controlled movement of the protective device.

According to an embodiment of the method, moving the protective device comprises a supplying an electric drive of the protective device with electrical energy from an electric storage element.

The electrical storage element may additionally supply further electrical consumers of the protective device with electrical energy, in particular a ventilation element, a control element and/or a disinfection element.

Due to the fact that moving the protective device comprises supplying an electric drive of the protective device with electrical energy from an electric storage element, the protective device may be moved particularly easy and with low personal expenses. Thereby, the protective device may be used particularly versatile.

An embodiment of the method comprises ventilating an interior space of the protective device.

Ventilating the interior space may take place by suppling the interior space with air or oxygen from a gas storage, for example from a pressure bottle filled with air or oxygen. Alternatively, or additionally, ventilating of the interior space may comprise creating a supply air flow of air from the environment into the interior space and/or an exhaust air flow of air from the interior space into the environment. Ventilating may comprise filtering of the air stream, i.e. of the supply air flow, the exhaust air flow or both air flows. Filtering may, for example, comprise passing through an activated carbon filter, a HEPA filter, or an electric filter. Additionally, filtering may comprise radiating the air stream with UV radiation. In particular situations, for example if the protective device is located in a secure environment, an unfiltered air stream may be provided although filtering means are provided.

Due to the fact that the method comprise ventilating the interior space, the method allows a passenger an extended stay in the protective device without pathogens being exchanged between the interior space and the environment.

According to an embodiment, the method comprises creating an overpressure and/or a negative pressure in the interior space of the protective device.

Creating an overpressure and/or a negative pressure may take place by creating an exhaust air flow that is stronger than a provided supply air flow, for example by fans of different power or by air ducts with different diameters or air resistance. Thereby, a negative pressure can be created in the interior space towards the environment. Thereby, it can be additionally avoided that air from the interior space reaches the environment, passing the ventilation element.

Due to the fact that the method comprises creating a negative pressure, can be better avoided that air from the interior space reaches the environment, passing the ventilation element. Because the method comprises creating an overpressure, it can be better avoided that air from the environment reaches the interior space, passing the ventilation element. Thereby, the protective device may better protect against pathogens.

According to an embodiment the method comprises remote-controlling the protective device.

Remote-controlling may comprise wireless sending and/or receiving of data, for example using mobile communication (also identified as WAN or wide area network), using local radio (also identified as wireless local area network or WLAN), using near radio (also identified as wireless personal area network or WPAN), and/or using directional radio. Therein, for example, control data may be received from hospital staff or from a control computer, with which the protective device may be remote-controlled.

Remote-controlling the protective device may comprise remote-controlling arbitrary functions of the protective device, in particular moving, ventilating, and disinfecting.

Due to the fact that the method comprises remote-controlling the protective device, the protective device may be used without extensive training of the passenger. Thereby, the protective device may be well utilized and safely operated.

According to an embodiment, the method comprises disinfecting the protective device.

Disinfecting comprises providing a disinfectant from a disinfection reservoir. The disinfectant may, for example, provided to jets that vaporize the disinfectant. Disinfecting comprises applying, e.g. vaporizing, disinfectant in the interior space of the protective device and/or on the exterior of the protective device, in particular on the exterior of the protective housing. Disinfecting may in particular comprise applying disinfectant in an area of the access opening.

Disinfecting may take place manually, for example by pumping and moving a flexible jet.

Disinfecting may take place mechanically, for example in a predefined angle and/or by a mechanical rotation of the jets. Disinfecting may be triggered at the touch of a button or remote-controlled. Disinfecting may take place automatically, for example based on a predefined schedule, upon opening of the access opening, and/or triggered based on a determined, estimated or calculated degree of contamination.

As the method comprises disinfecting, it can be ensured that the protective device is free of pathogens. Thereby, a safe operation of the method can be ensured.

Regarding advantages, embodiments and details of the method and its embodiments, it is referred to the previous description of the related features of the device.

The protective device and the method are particularly suited to be used to protect a patient in an environment of the protective device against pathogens of a passenger of the protective device. The protective device is a protective device according one of the examples described above.

The protective device and the method are additionally suited to be used to protect a passenger in the protective device against pathogens of a patient in the environment of the protective device. The protective device is a protective device according to one of the examples described above.

Regarding advantages, embodiments and details of the use and its embodiments, it is referred to the previous description of the related features of the device and method.

A preferable embodiment of the disclosure is, for example, described in conjunction with the accompanying figures, which show:

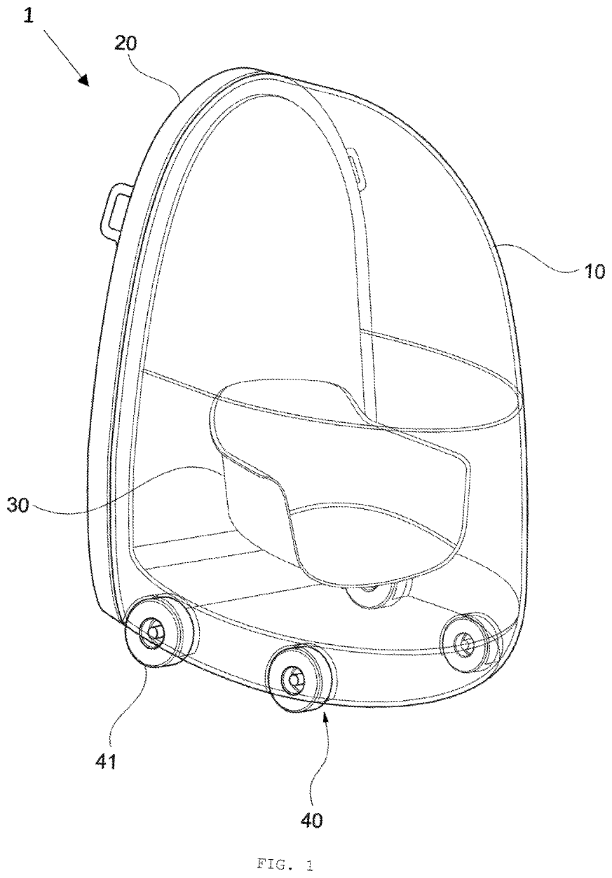

FIG. 1 a perspective view of a protective device according to an embodiment;

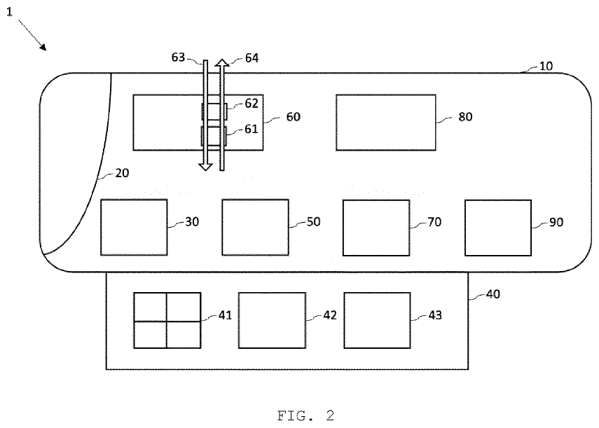

FIG. 2 a schematical view of a protective device according to an embodiment; and

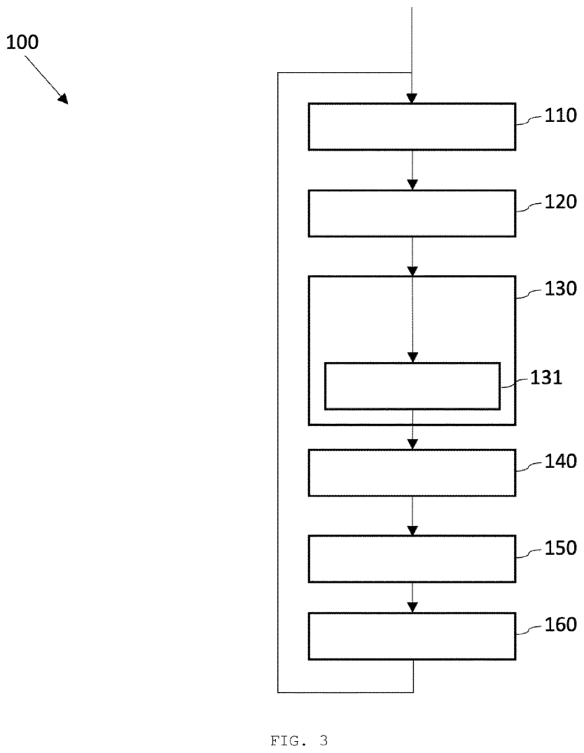

FIG. 3 a block diagram of a method according to an embodiment.

FIG. 1 shows a perspective view of a protective device 1 according to an embodiment.

The protective device 1 comprises a protective housing 10 that is mostly translucent, i.e., semi-transparent.

The protective housing 10 is, for example, made from an acrylic glass. Thereby, the protective housing 10 has particularly good optical properties, for example a low optical distortion and reflection. Thereby, the protective housing 10 may ease and improve communication of a passenger with persons in the environment of the protective housing 10.

The acrylic glass is partly tinted, wherein the acrylic glass may be tainted darker in an upper and a lower part as in an area of the eye level of the passenger. Thereby, the protective housing 10 may provide a protection of the passenger against heat radiation, for example sun radiation, and against cooling. Furthermore, privacy of the passenger may be improved.

The protective housing 10 has a rounded, convex form.

For example, the protective housing 10 has a generally semi-oval basic form. On a backside the protective housing 10 may be formed generally flat. Furthermore, a bottom side of the protective housing 10 may be generally flat. The backside and the bottom side are connected by a right angle, wherein the protective housing 10 is rounded between the backside and the bottom side. The rest of the protective housing 10 has an oval form. The protective housing does not have any edges or corners in general.

Thereby, the protective device 1 may be particularly compact and easy to clean, and ease and improve a communication of a passenger with persons in the environment of the protective housing 10.

On the backside the protective housing 10 has an access opening 20. The access opening 20 may be openable to a side. Thereby, entry and exit of the passenger is eased and the access opening 20 is space saving. Thereby, the protective device 1 may also be used in a narrow environment. The access opening 20 may alternatively or additionally be openable towards the bottom.

Thereby, a door of the access opening 20 may rest on the floor in an opened state and be used as a ramp. Thereby, for example, a passenger with a wheelchair may be wheeled through the access opening 20 into the protective device 1.

In alternative embodiment not shown, the access opening 20 may be located on a front side of the protective housing 10. In an example, the access opening 20 may account for a large part of the protective housing 10, for example more than 50% of the protective housing 10. The access opening 20 may, in this case, be openable to the side or upwards.

The protective housing 10 defines an interior space of the protective device 1. In the interior space there is arranged a seating element 30.

The seating element 30 may be mounted rotatably on a center axis, which is not shown. In an example, the seating element 30 may be rotatable by 360°. Alternatively, the seating element 30 may be rotatable by 180°. The seating element 30 may snap in in one or more positions, for example in a position facing the backside of the protective housing 10 and in a position facing the front side of the protective housing 10. Thereby, the seating element 30 may be rotated in the direction of the backside of the protective housing 10 when the access opening 20 is opened and be rotated in the direction of the front side of the protective housing 10 when the access opening 20 is closed, for example automatically. Thereby, the protective device 1 may be entered and exited particularly easily and comfortably.

In alternative embodiments not shown, the protective housing 10 may have a drop-shaped basic form. In alternative embodiments not shown, the protective housing 10 may also have an egg-shaped basic form. In alternative embodiments not shown, the protective housing 10 may also have a cuboid basic shape. In alternative embodiments not shown, the protective housing 10 may also have a wedge-shaped basic form.

On the bottom side of the protective housing 10 there is shown a chassis 40 for moving of the protective device 1. The chassis 40 comprises four wheels 41.

The protective device 1 seals the interior space in an airtight manner against an environment in a closed state of the access opening 20. That means that an exchange of air between the interior space and the environment is minimized or excluded.

In an example, the air tightness of the protective device 1 is better than a leakage rate of 2.21 m³/m²/h at 20 Pa. That means that at a differential pressure of 20 Pa less than 2.21 m³ air per 1 m² surface is exchanged in one hour. In this or in a further example the air tightness of the protective device 1 is better than a leakage rate of 3.78 m³/m²/h at 50 Pa.

In an example, the air tightness of the protective device 1 is better than a leakage rate of 0.43 m³/m²/h at 20 Pa. In this or in a further example the air tightness of the protective device 1 is better than a leakage rate of 0.80 m³/m²/h at 50 Pa.

The air tightness of the protective device 1 is in particular based on an airtight closing of the access opening 20. The access opening 20 consists of a hatch that may also be identified as a door or a flap, and a receiving portion. In a closed state the hatch engages with the receiving portion. The hatch and/or the receiving portion comprise a seal, in particular a rubber lip. In a closed state the hatch is pressed into the receiving portion, such that the seal does not allow for an exchange of air.

The contact pressure force between the hatch and the receiving portion is maintained by a lever lock that locks the access opening 20.

In an alternative embodiment that is not shown, the contact pressure force between the hatch and the receiving portion is maintained by a snap lock and/or a screw lock that locks the access opening 20.

FIG. 2 shows a schematic view of a protective device 1 according to an embodiment. In a further embodiment not shown, the locking mechanism may be operated electrically.

The protective device 1 comprises a protective housing 10 that defines an interior space of the protective device 1. The protective device 1 seals the interior space in an airtight manner against its environment in a closed state.

The protective device 1 further comprises a closable access opening 20 that is configured as a part of the protective housing 10 in this embodiment. The access opening 20 may be pivoted around a hinge to allow for entering and exiting the interior space.

The protective device 1 further comprises a seating element 30 that allows seating for a passenger in the interior space. The seating element 30 is arranged in the interior space.

The protective device 1 further comprises a chassis 40 to move the protective device 1. The chassis 40 is arranged on a bottom side of the protective housing.

The chassis 40 may be embodied as a skateboard and may be releasably connected with the protective housing 10. A skateboard is a flat structure that comprises a chassis and a support structure that may receive a setup. Thereby, the chassis may be easily exchanged. In an example, a passives skateboard may be changed to an electrically powered skateboard, or the other way around.

In another example, the chassis 40 may be configured integral, inseparably connected and/or in one piece with the protective housing 10.

The chassis 40 may comprise multiple wheels 41. In the embodiment shown in FIG. 2, the chassis comprises four wheels 41.

The chassis 40 further comprises a steering 42 that is configured to control a steering angle of the wheels. Thereby, the protective device 1 may be steered.

The chassis 40 further comprises an electric drive 43. The electrical drive may comprise an electric motor and wheel axis that is driven by the electric motor. The wheel axis in this case is exemplarily connected with two wheels 41 and transmits the torque generated by the electric motor to the two wheels.

Alternatively, or additionally, the electrical drive may comprise one or more electrical motors that are each arranged in or at a wheel 41. Therein, the principle of a wheel hub motor is applied. The electrical drive may also be configured as any other suitable electrical drive.

The electrical drive may contain, for example, separate electric motors, for example wheel hub motors, for each back and/or front wheel. Therein, the wheels 41 may in particular be configured such that they do not have a connecting axis. Thereby, maneuvering is particularly easy and a particularly compact design of the protective device 1 is achieved.

In an example, exclusively two front wheels or exclusively two rear wheels are driven by the electric drive. This allows for a particular favorable weight distribution in relation to the weight of the passenger.

Alternatively, all four wheels 41 are driven by the electric drive. Thereby, off-road capabilities as well as redundancy is achieved.

In particular, the driven wheels 41 may be driven independently of each other, so as to achieve easy maneuvering and a small curve radius. Furthermore, the front wheels may be configured smaller than the rear wheels or the rear wheels may be configured smaller than the front wheels, to ensure for an easy entry and a spacious legroom for the passenger. In an example, the size difference between front wheels and rear wheels is between 15% and 30%. In another example, the size difference between front wheels and rear wheels is 24%.

The electrical drive 43 is supplied with electrical energy by an electrical storage element 50 of the protective device 1. In an alternative embodiment the electrical drive 43 may be supplied with electrical energy from an additional electrical drive storage element that is arranged in the chassis 40. In an alternative embodiment the electrical storage element 50 may be arranged in the chassis 40.

The electrical storage element 50 comprises, for example, an accumulator battery, i.e., an arrangement of rechargeable galvanic elements. The electrical storage element 50 supplies the protective device 1 with electrical energy, i.e. electrical consumers of the protective device 1, such as, for example, the seating element 30, a ventilation element 60 as will be later described, a control element 70 as will be later described, and/or a disinfection element 80 as will be later described.

The electrical storage element 50 may be redundant. That means that the electrical storage element 50 comprises at least two electrical energy storages, for example two accumulator batteries. Each of the electric energy storages is separately electrically connected with the electric consumers of the protective device 1. Thereby, a functionality of the protective device 1 may be ensured, even if one of the electric energy storages or an electric connection fails.

The electrical storage element 50 may be supplied with energy through an electrical connection with an energy source in the environment. In an example, the electrical storage element 50 may be charged through a socket, using a cable. Alternatively, or additionally, the electrical storage element 50 may be charged wirelessly through an inductive charging device using an inductive energy recipient. Alternatively, or additionally, the electrical storage element 50 may be charged by a solar module that, for example, comprises solar cells arranged on or in the protective device 1.

The protective device 1 further comprises a ventilation element 60. The ventilation element 60 is configured to provide an air stream to the interior space. Thereby, an exchange of air between the interior space and the environment may be controlled or prevented.

In an alternative embodiment not shown the ventilation element 60 may comprise a gas storage, for example from a pressure bottle filled with air or oxygen. In this case, an exchange of air between the interior space and the environment may be prevented for an extended period of time. A supply of oxygen to a passenger of the protective device 1 may alternatively be ensured by oxygen carried in the gas storage.

The present ventilation element 60 creates an air stream between the environment of the protective device 1 and the interior space of the protective device 1, for example through a ventilator. Furthermore, the ventilation element 60 comprises a filter element 61. The filter element 61 is configured to filter the air stream, i.e., to degerminate, to disinfect and/or to sterilize. The filter element 61 may comprise a HEPA filter, wherein the air stream is directed through the HEPA-Filter. The filter element 61 can further comprise an ultraviolet (UV) radiation source that irradiates the air stream with UV light in a section. The filter element 61 filters supply air flow 63 from the environment of the protective device 1 to the interior space of the protective device 1. Thereby, a passenger in the interior space may be protected against pathogens from the environment. The filter element 61 further filters an exhaust air flow 64 from the interior space of the protective device 1 to the environment of the protective device 1. Thereby, the environment, for example a patient in the environment of the protective device 1, may be protected against pathogens from the interior space.

In alternative embodiments not shown the filter element 61 may exclusively filter an exhaust air flow 64. In alternative embodiments not shown the filter element 61 is adapted to exclusively filter a supply air flow 63.

The ventilation element 61 is further configured to create an overpressure and/or a negative pressure in the interior space. In an example, the ventilation element 61 may be configured, to provide an exhaust air flow 64 that is stronger than a supply air flow 63, for example by using ventilators of different power or by using air ducts with different diameters. Thereby, it is possible to provide a negative pressure towards the environment in the interior space. Thereby, it can be additionally avoided that air from the interior space reaches the environment, passing the ventilation element 60.

The ventilation element 60 further comprises an air conditioning element 62. The air conditioning element 62 is adapted to transfer heat from the supply air flow 63 to the exhaust air flow 64, for example using a heat pump. Thereby, the interior space may be cooled compared to the environment. The air conditioning element 62 may, vice versa, transfer heat from the exhaust air flow 64 to the supply air flow 63. Thereby, the interior space may be heated compared to the environment. The air conditioning element 62 may further be configured to condition the supply air flow 63, for example to raise or reduce humidity of the supply air flow 63. Thereby, a comfortable and/or healthy air climate may be created in the interior space. Thereby, the protective device 1 may be particularly suitable for passengers with respiratory diseases, or particularly weakened passengers. Furthermore, the protective device 1 may be suitable for application in special environments, for example particularly hot or cold environments.

The protective device 1 further comprises a control element 70.

The control element 70 is configured to remote-control the protective device 1. The control element 70 may be configured to wirelessly send and/or receive data, for example through a module for mobile communication (also identified as WAN or wide area network), a module für local radio (also identified as wireless local area network or WLAN), a module for near radio (also identified as wireless personal area network or WPAN), and/or a module for directional radio. The control element exchanges data with a remote control device that for example may be, for example, provided in the environment of the protective device 1. The remote-control device may be operated, for example, by hospital staff or controlled automatically by a control computer.

The control element 70 may be configured, to remote-control arbitrary functions of the protective device 1, in particular the chassis 40, the ventilation element 60, and disinfection element 80, as described below.

For example, the chassis 40 may be remote-controlled by a person that is directly in the line of sight of the protective device 1, for example by a nurse. In a further example, the chassis 40 may be remote-controlled by a person that has vision of a driveway of the protective device 1 by use of a camera, for example by a service employee in a control room distanced from the protective device, such as, for example, a call center. In a further example, the chassis 40 may be remote-controlled by a computer, for example an autonomous driving computer.

For example, the ventilation element 60 may be switched on, switched of or adjusted through the control element 70 in a remote-controlled manner. Thereby, it is possible to adjust to provide a particularly filtered air stream when entering a secure area, and/or to adjust to provide a less filtered air stream when leaving the secure area.

The protective device 1 further comprises a disinfection element 80. The disinfection element 80 is configured, to disinfect a part of the protective device 1 or the whole protective device 1.

The disinfection element 80 comprises one or more jets that are connected to a disinfection reservoir. The disinfection element 80 provides disinfectant from the disinfection reservoir to the jets, whereby the disinfectant is sprayed. The disinfection element 80 applies disinfectant in the interior space of the protective device 1 and/or on the exterior of the protective device 1, in particular on the exterior of the protective housing 10. The disinfection element 80 furthermore applies disinfectant in an area of the access opening 30.

In an alternative embodiment, which is not shown, the disinfection element 80 is configured, to apply disinfectant manually, for example by using a pump device and a flexibly moveable jet.

In the embodiment as shown in FIG. 2, the disinfection element is configured to apply disinfectant mechanically, for example in a predetermined angle and/or by a mechanical rotation of the jets. Application of disinfectant may be triggered on the touch of a button or remote-controlled, in particular remote-controlled via control element 70. The disinfection element 80 can apply disinfectant automatically, for example based on a predefined schedule, upon opening of the access opening and/or on a determined, estimated or calculated degree of contamination.

FIG. 3 shows a block diagram of a method according to an embodiment. The method 100 starts with a method step of providing 110 a protective device, in particular a protective device according to one of the examples as described above, to a passenger.

In a further method step the method comprises closing 120, in an airtight manner, the protective device.

In a further method step the method comprises ventilating 130 the interior space.

Ventilating 130 the interior space comprises den further method step of creating 131 a negative pressure or an overpressure in the interior space.

In a further method step the method comprises moving 140 the protective device.

In a further method step the method comprises remote-controlling 150 the protective device. In particular, remote-controlling 150 may comprise moving 140.

In a further method step the method comprises disinfecting 160 the protective device.

After disinfecting 160 the protective device the method may return to the method step of providing 110 the protective device.

In a further method step of the method not shown, leak tightness of the protective device may be checked during operation.

In a further method step of the method not shown, the air quality on the interior may be checked for pathogens during use.

In alternative embodiments, individual method steps of the method 1 may be omitted, repeated and/or executed in a different order of the method.

The protective device to protect against pathogens can particularly suitable realized in one of the following example embodiments:

Example 1: Protective device to protect against pathogens, comprising:
a protective housing that defines an interior space, and
a closable access opening to enter the interior space.

Example 2: Protective device to protect against pathogens, comprising:
a protective housing that defines an interior space, and
a seating element that allows a passenger to sit in the interior space.

Example 3: Protective device to protect against pathogens, comprising:
a protective housing that defines an interior space, and
a chassis for moving the protective device.

Example 4: Protective device to protect against pathogens, comprising:
a protective housing that defines an interior space, wherein the protective device den interior space in an airtight manner against an environment seals.

Example 5: Protective device to protect against pathogens, comprising:
a protective housing that defines an interior space, a closable access opening to enter the interior space, and
a seating element that allows a passenger to sit in the interior space.

Example 6: Protective device to protect against pathogens, comprising:
a protective housing that defines an interior space, a closable access opening to enter the interior space, and
a seating element that allows a passenger to sit in the interior space,
wherein the protective device in a closed state den interior space in an airtight manner against an environment seals.

Example 7: Protective device according to one of the previous examples, wherein the chassis comprises an electric drive.

Example 8: Protective device according to one of the previous examples, further comprising:
an electrical storage element, configured to supply the electric drive with energy.

Example 9: Protective device according to one of the previous examples, wherein the protective device is designed hygienic.

Example 10: Protective device according to one of the previous examples, wherein the protective housing comprises an anti-bacterial and/or an anti-viral coating.

Example 11: Protective device according to one of the previous examples, further comprising:
a ventilation element, configured to provide an air stream to the interior space.

Example 12: Protective device according to one of the previous examples, wherein the protective device is configured to create an overpressure and/or a negative pressure in the interior space.

Example 13: Protective device according to one of the previous examples, further comprising:
a control element, configured to control the protective device.

Example 14: Protective device according to one of the previous examples, further comprising:
a disinfection element that is configured for disinfecting the protective device.

### LIST OF REFERENCE NUMERALS

1 protective device
10 protective housing
11 coating
20 access opening
30 seating element
40 chassis
41 wheel
42 steering
43 electrical drive
50 electrical storage element
60 ventilation element
61 filter element
62 air conditioning element
63 supply air flow
64 exhaust air flow
70 control element
80 disinfection element
100 method
110 method step of providing
120 method step of closing in an airtight manner
130 method step of ventilating
131 method step of creating
140 method step of moving
150 method step of remote-controlling
160 method step of disinfecting

1-15. (canceled)

16. Protective device to protect against pathogens, comprising:
a protective housing that defines an interior space,
a closable access opening to enter the interior space,
a seating element that allows at least one passenger to sit in the interior space, and
a chassis for moving the protective device,
wherein the protective device, in a closed state, seals the interior space in an airtight manner against an environment.

17. Protective device according to claim 16, wherein the chassis comprises an electric drive, and the protective device further comprising:
an electrical storage element, configured to supply the electric drive with energy.

18. Protective device according to claim 16, wherein the protective device is configured to move autonomously.

19. Protective device according to claim 16, wherein the protective housing comprises an anti-bacterial and/or an anti-viral coating.

20. Protective device according to claim 16, further comprising:
a ventilation element, configured for providing an air stream to the interior space.

21. Protective device according to claim 20, wherein the ventilation element is configured to create an overpressure and/or a negative pressure in the interior space.

22. Protective device according to claim 16, wherein the protective device is configured to be remote-controlled.

23. Protective device according to claim 16, further comprising an emergency element.

24. Protective device according to claim 16, further comprising an intercom system.

25. Protective device according to claim 16, further comprising at least one pass-through element.

26. Protective device according to claim 25, wherein the at least one pass-through element comprises an invertible glove.

27. Protective device according to claim 16, further comprising:
a control element, configured for controlling the protective device.

28. Protective device according to claim 27, wherein the control element is arranged at the outer side of the protective device.

29. Protective device according to claim 27, wherein the control element is configured as a joystick.

30. Protective device according to claim 16, further comprising:
a disinfection element, configured for disinfecting the protective device.

31. Protective device according to claim 16, wherein the access opening is located on a front side of the protective housing.

32. Protective device according to claim 16, further comprising at least one sensor being configured to measure vital data of at least one passenger of the protective device.

33. Protective device according to claim 16, further comprising at least one sensor being configured to measure the air on the interior and/or the exterior of the protective device.

34. Protective device according to claim 16, wherein the seating element has a seating surface that is adjustable in height.

35. Method to protect against pathogens, comprising
providing a protective device to a passenger,
closing the protective device in an airtight manner, and
moving the protective device.
